(19) United States
(12) Patent Application Publication
(10) Pub. No.: US 2011/0087301 A1
(43) Pub. Date: Apr. 14, 2011

(54) IMPLANTABLE DEVICE WITH HEMODYNAMIC SUPPORT OR RESUSCITATION THERAPY

(76) Inventors: Dan Li, Shoreview, MN (US); Allan C. Shuros, St. Paul, MN (US); Arjun Sharma, St. Paul, MN (US); Stephen Ruble, Lino Lakes, MN (US)

(21) Appl. No.: 12/892,298

(22) Filed: Sep. 28, 2010

Related U.S. Application Data
(60) Provisional application No. 61/250,308, filed on Oct. 9, 2009.

(51) Int. Cl. A61N 1/365; A61N 1/36; A61N 1/39

(52) U.S. Cl. 607/5; 607/42; 607/14

(57) ABSTRACT

An apparatus comprises an implantable sensor, a stimulation circuit, and a controller. The implantable sensor is configured to provide a sensor signal representative of hemodynamic function of a subject. The stimulation circuit is configured to provide electrical simulation energy to an implantable electrode. The controller is communicatively coupled to the stimulation circuit and the implantable sensor and includes a hemodynamic monitor module. The hemodynamic monitor module is configured to detect an episode of reduced hemodynamic capacity in a subject using the sensor signal. In response to the detected episode, the controller is configured to initiate delivery of the electrical stimulation energy to artificially induce at least one of deep ventilation or rapid ventilation in the subject. The hemodynamic monitor module is configured to obtain a measure of hemodynamic performance after delivery of the electrical stimulation energy.

### CROSS-REFERENCE TO RELATED APPLICATION

This application claims the benefit of U.S. Provisional Application No. 61/250,308, filed on Oct. 9, 2009, under 35 U.S.C. §119(e), which is incorporated herein by reference in its entirety.

### BACKGROUND

Implantable medical devices (IMDs) include devices designed to be implanted into a patient. Some examples of these devices include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients or subjects using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Some IMDs include one or more sensors to monitor different aspects of the patient's cardiovascular system. These sensors enable the IMD to detect when the subject is experiencing an episode of reduced hemodynamic capacity. The capacity of the hemodynamic system of the subject may be reduced due to development of breathing disorder or due to an episode of hemodynamic compromise. Hemodynamic compromise includes any condition that impedes proper blood flow (a severe example is a heart attack). A cardiac arrhythmia, such as a tachyarrhythmia, may also reduce capacity of the hemodynamic system. Tachyarrhythmia includes abnormally rapid heart rate, or tachycardia, including ventricular tachycardia (VT) and supraventricular tachycardia (SVT). Tachyarrhythmia also includes rapid and irregular heart rate, or fibrillation, including ventricular fibrillation (VF). Monitoring for reduced hemodynamic capacity of the patient can lead to providing prompt proper treatment of the condition.

### Overview

This document relates generally to systems, devices, and methods for providing resuscitation therapy to a patient or subject. In particular, a device artificially induces at least one of deep ventilation or rapid ventilation in the subject to relieve episodes of reduced hemodynamic capacity.

Example 1 can include an implantable sensor, a stimulation circuit, and a controller. The implantable sensor is configured to provide a sensor signal representative of hemodynamic function of a subject. The stimulation circuit is configured to provide electrical simulation energy to an implantable electrode. The controller is communicatively coupled to the stimulation circuit and the implantable sensor and includes a hemodynamic monitor module. The hemodynamic monitor module is configured to detect an episode of reduced hemodynamic capacity in a subject using the sensor signal. In response to the detected episode, the controller is configured to initiate delivery of the electrical stimulation energy to artificially induce at least one of deep ventilation or rapid ventilation, such as a cough or gasp, in the subject. The hemodynamic monitor module is configured to obtain a measure of hemodynamic performance after delivery of the electrical stimulation energy.

In example 2, the subject matter of example 1 can optionally include an implantable electrode communicatively coupled to the electrical stimulation circuit, wherein the implantable electrode is configured for placement on or near at least one of a phrenic nerve or a vagus nerve, and wherein the controller is configured to initiate electrical stimulation of the phrenic nerve or the vagus nerve to artificially induce the deep ventilation or rapid ventilation.

In example 3, the subject matter of any one of examples 1 or 2 can optionally include an implantable electrode communicatively coupled to the electrical stimulation circuit, wherein the implantable electrode is configured for placement on or near a diaphragm of the subject, and wherein the controller is configured to initiate delivery of electrical stimulation energy to the diaphragm to artificially induce the deep ventilation or rapid ventilation.

In example 4, the subject matter of any one of examples 1-3 can be optionally configured to initiate delivery of the electrical stimulation energy to artificially induce at least one of a cough or gasp by the subject in response to the detected episode.

In example 5, the subject matter of any one of examples 1-4 can include a therapy circuit communicatively coupled to the controller and configured to provide anti-tachyarrhythmia therapy that includes at least one of: anti-tachycardia pacing (ATP); cardioversion shock therapy; or defibrillation shock therapy. The subject matter can also optionally include a cardiac signal sensing circuit configured to provide a sensed cardiac signal representative of cardiac depolarization events of the subject. The subject matter can further optionally be configured to detect an episode of tachyarrhythmia using the sensed cardiac signal, and to initiate electrical stimulation to induce the artificial hyperventilation prior to initiating anti-tachyarrhythmia therapy.

In example 6, the subject matter of example 5 can be optionally configured to reconfirm the tachyarrhythmia after the electrical stimulation to artificially induce the deep ventilation or rapid ventilation is delivered and prior to the initiation of the tachyarrhythmia therapy.

In example 7, the subject matter of any one of examples 1-6 can optionally include a therapy circuit communicatively coupled to the controller, and the subject matter is optionally configured to provide anti-tachyarrhythmia therapy that includes at least one of: anti-tachycardia pacing (ATP); cardioversion shock therapy; or defibrillation shock therapy. The subject matter can further optionally include a cardiac signal sensing circuit configured to provide a sensed cardiac signal representative of cardiac depolarization events of the subject, and the subject matter can be further optionally configured to detect an episode of tachyarrhythmia using the sensed cardiac signal, and to initiate the electrical stimulation to artificially induce the deep ventilation or rapid ventilation after anti-tachyarrhythmia therapy is delivered.

In example 8, the subject matter of example 7 can be optionally configured to initiate the electrical stimulation to artificially induce the deep ventilation or rapid ventilation when at least one of: the episode of tachyarrhythmia is sustained for a time duration that exceeds a specified time duration threshold; or the anti-tachyarrhythmia therapy includes shock therapy, and the tachyarrhythmia is sustained after delivery of a number of shocks that exceeds a threshold number of shock therapy deliveries.

In example 9, the subject matter of any one of examples 1-8 can optionally include at least one of: an activity sensor configured to provide a sensor signal representative of chest muscle activity; and a transthoracic impedance sensor to provide a sensor signal representative of transthoracic impedance. The subject matter can be optionally configured to determine an intensity of the induced ventilation using the provided sensor signal and to adjust the electrical stimulation energy to artificially induce the deep ventilation or rapid ventilation according to the determined intensity.

In example 10, the subject matter of any one of examples 1-9 can be optionally configured to, in response to the measure of hemodynamic performance, adjust the electrical stimulation to change an intensity of the deep ventilation or the rapid ventilation; or select an alternate implantable electrode to deliver the electrical stimulation.

In example 11, the subject matter of any one of examples 1-10 can be optionally configured to detect, using the sensor signal provided by the implantable sensor, at least one of: a decrease in respiration tidal volume; or an onset of hemodynamic compromise.

In example 12, the subject matter of any one of examples 1-11 can optionally comprise detecting, using an implantable medical device (IMD), an episode of reduced hemodynamic capacity in a subject, artificially inducing at least one of deep ventilation or rapid ventilation in the subject using electrical stimulation energy provided by the IMD in response to detecting the episode of reduced hemodynamic capacity, and obtaining a measure of hemodynamic performance after delivery of the electrical stimulation energy.

In example 13, the subject matter of any one of examples 1-12 can optionally comprise, according to the measure of hemodynamic performance, at least one of: adjusting the electrical stimulation provided by the IMD to change the strength of the deep ventilation or the rapid ventilation; or changing at least one electrode used in providing the electrical stimulation.

In example 14, the subject matter of any one of examples 1-13 can optionally be configured such that detecting an episode of reduced hemodynamic capacity includes detecting an episode of tachyarrhythmia, artificially inducing one or both of the deep ventilation or rapid ventilation includes inducing one or both of the deep ventilation or rapid ventilation prior to providing anti-tachyarrhythmia therapy with the IMD, and the anti-tachyarrhythmia therapy optionally includes at least one of: anti-tachycardia pacing (ATP); cardioversion shock therapy; or defibrillation shock therapy.

In example 15, the subject matter of example 14 optionally comprises reconfirming the tachyarrhythmia after artificially inducing one or both of the deep or rapid ventilation and prior to providing the anti-tachyarrhythmia therapy.

In example 16, the subject matter of any one of examples 1-15 can optionally be configured such that detecting an episode of reduced hemodynamic capacity includes detecting an episode of tachyarrhythmia, and artificially inducing the deep or rapid ventilation includes artificially inducing one or both of the deep ventilation or rapid ventilation after providing anti-tachyarrhythmia therapy with the IMD.

In example 17, the subject matter of example 16 can optionally be configured such that artificially inducing deep or rapid ventilation after providing anti-tachyarrhythmia therapy includes artificially inducing one or both of the deep or rapid ventilation when at least one of: the episode of tachyarrhythmia is sustained for a time duration that exceeds a specified time duration threshold, or the anti-tachyarrhythmia therapy includes shock therapy, and the tachyarrhythmia is sustained after delivery of a number of shocks that exceeds a threshold number of shock therapy deliveries.

In example 18, the subject matter of any one of examples 1-17 can optionally be configured such that artificially inducing one or both of deep ventilation or rapid ventilation includes artificially inducing at least one of a cough or gasp by the subject.

In example 19, the subject matter of any one of examples 1-18 can optionally comprise determining an intensity of the artificially induced deep ventilation or rapid ventilation; and adjusting the electrical stimulation provided by the IMD to change the intensity of the deep ventilation or rapid ventilation.

In example 20, the subject matter of any one of examples 1-19 can optionally be configured such that obtaining a measure of hemodynamic performance includes obtaining a measure of at least one of: arterial pressure; cardiac stroke volume; coronary perfusion; or cerebral perfusion.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of an example of portions of a system that includes an IMD.

FIG. 2 is an illustration of an example of an IMD implanted in a thorax region of a patient.

FIG. 3 shows a flow diagram of an example of a method of artificially inducing ventilation in a subject using an IMD.

FIG. 4 shows a block diagram of portions of an example of a device that artificially induces ventilation in a subject.

### DETAILED DESCRIPTION

An IMD may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features described herein, but may be implemented to include selected features that provide for unique structures or functionality. Such a device may be implemented to provide a variety of therapeutic or diagnostic functions.

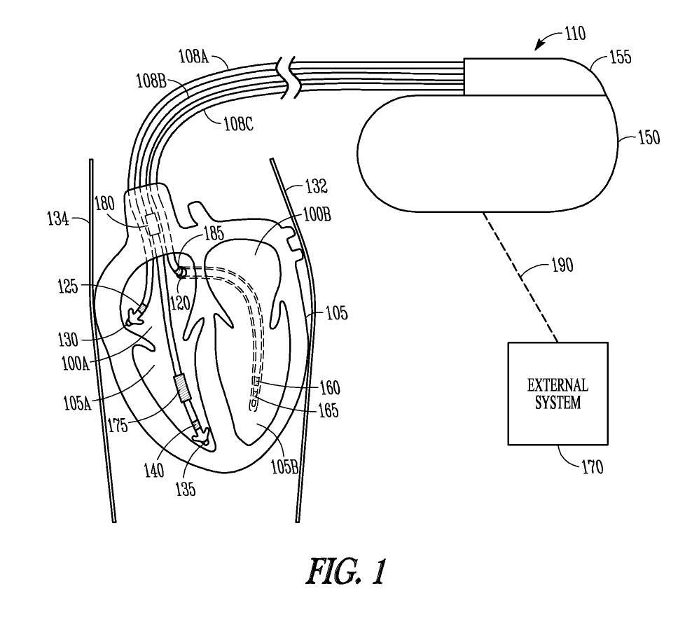

FIG. 1 is an illustration of portions of a system that uses an IMD 110. Examples of IMD 110 include, without limitation, a pacer, a defibrillator, a cardiac resynchronization therapy (CRT) device, or a combination of such devices. The system also typically includes an IMD programmer or other external device 170 that communicates wireless signals 190 with the IMD 110, such as by using radio frequency (RF) or other telemetry signals.

The IMD 110 is coupled by one or more leads 108A-C to heart 110. Cardiac leads 108A-C include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A.

Right ventricular (RV) lead 108B includes one or more electrodes, such as tip electrode 135 and ring electrode 140, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 105. Such electrodes typically have larger surface areas than pacing electrodes in order to handle the larger energies involved in defibrillation. Lead 108B optionally provides resynchronization therapy to the heart 105. Resynchronization therapy is typically delivered to the ventricles in order to better synchronize the timing of depolarizations between ventricles.

The IMD 110 may include a third cardiac lead 108C attached to the IMD 110 through the header 155. The third cardiac lead 108C includes ring electrodes 160 and 165 placed in a coronary vein lying epicardially on the left ventricle (LV) 105B via the coronary vein. The third cardiac lead 108C may include a ring electrode 185 positioned near the coronary sinus (CS) 120.

Lead 108B may include a first defibrillation coil electrode 175 located proximal to tip and ring electrodes 135, 140 for placement in a right ventricle, and a second defibrillation coil electrode 180 located proximal to the first defibrillation coil 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to an electrode formed on the hermetically-sealed IMD housing or can 150. This improves defibrillation by delivering current from the RV coil 175 more uniformly over the ventricular myocardium. In some examples, the therapy is delivered from the RV coil 175 only to the electrode formed on the IMD can 150.

Note that although a specific arrangement of leads and electrodes are shown the illustration, the present methods and systems will work in a variety of configurations and with a variety of electrodes. Other forms of electrodes include meshes and patches which may be applied to portions of heart 105 or which may be implanted in other areas of the body to help “steer” electrical currents produced by IMD 110. The IMDs may be configured with a variety of electrode arrangements, including transvenous, endocardial, or epicardial electrodes (e.g., intrathoracic electrodes), or subcutaneous, non-intrathoracic electrodes, such as can, header, or indifferent electrodes, or subcutaneous array or lead electrodes (e.g., non-intrathoracic electrodes). Monitoring of electrical signals related to cardiac activity may provide early, if not immediate, diagnosis of cardiac disease.

An IMD 110 may include one or more sensors. The sensors provide a time-varying electrical signal that is related to physiologic cardiovascular events of a subject. A non-exhaustive list of examples of such sensors include a cardiac signal sensing circuit, an intracardiac impedance sensing circuit, a transthoracic impedance sensing circuit, a blood pressure sensor, a blood flow sensor, a blood gas sensor, a chemical sensor, a heart sound sensor, a posture sensor, and an activity sensor. In some examples, the IMD 110 communicates with a sensor external to the IMD 110. The signals provided by the sensors may be used to detect an episode of reduced hemodynamic capacity that a patient or subject is experiencing or has experienced.

As explained previously, an example of reduced hemodynamic capacity is an episode of tachyarrhythmia, such as VF, VT, or SVT. Artificially inducing one or both of deep ventilation and rapid ventilation in a patient, such as artificially inducing a gasp or cough, during an episode of VF can provide benefits to the subject during an episode of reduced hemodynamic capacity. This inducing of a cough or gasp is sometimes called resuscitation therapy, or artificially induced hyperventilation. If the episode includes VF, these benefits include an increase in hemodynamic performance. For example, a cough or a gasp by the subject may increase pulmonary gas exchange, increase blood return in the venous system, increase aortic pressure, and increase coronary perfusion pressure. A gasp induced during VF may also increase contractility of the heart. A gasp may also benefit cerebral blood circulation. For example, a gasp by the subject may increase the subject's carotid blood flow, cerebral perfusion pressure, and may increase cerebral microcirculatory blood flow velocity and duration of flow. If the episode includes hypotensive VT, inducing a cough during the episode, may increase mean arterial pressure of the subject during the episode.

A subject may benefit from an induced cough or gasp during other types of episodes of reduced hemodynamic capacity. For example, during a decrease in respiration tidal volume, inducing a cough or gasp may provide an increase in blood oxygenation. In another example, during an episode of hemodynamic compromise, such as ischemia, inducing a gasp may increase the subject's chance for survival.

Artificially inducing one or both of deep ventilation and rapid ventilation using an IMD can provide hemodynamic support for the subject when a need for such support is detected through sensors of the IMD. Gasps or coughs can be artificially induced by using the IMD 110 to stimulate one or more of the subject's phrenic nerves and the subject's vagus nerve. In some examples, gasps or coughs can be artificially induced by directly stimulating the subject's diaphragm. In some examples, the IMD 110 can be used to selectively stimulate the subject's thoracic or chest muscles to enhance inhalation after the gasp or cough. After delivery of stimulation energy to artificially induce one or more of deep ventilation and rapid ventilation, the sensors of the IMD may also provide feedback to allow adjustment of the artificially induced ventilation, such as intensity and timing of a cough or gasp.

FIG. 1 shows the left phrenic nerve 132 and a right phrenic nerve 134. The phrenic nerves run from the subject's neck area to the subject's diaphragm. The left phrenic nerve 130 runs near the left epicardium. Electrodes of LV lead 108C may be used to stimulate the left phrenic nerve 132 to induce the gasp or cough. Electrodes of the RV lead 108B may be used to stimulate the right phrenic nerve 134. In some examples, the SVC coil electrode 180 can be used to stimulate the right phrenic nerve 134. Bilateral stimulation of the left and right phrenic nerves can be provided using both the RV lead 120 and the LV lead 125. Other electrodes or electrode combinations may be useful in artificially inducing the deep ventilation and the rapid ventilation.

To find an optimum combination of electrodes (i.e., vector) to use in stimulating the phrenic nerve, different vectors may be tested at time of implant. In some examples, the IMD 110 is programmed to scan through different available vectors to find the vector that induces the strongest phrenic nerve stimulation (PNS) effect. Feedback provided by sensors such as those described herein enable the IMD 110 to determine the strongest PNS effect.

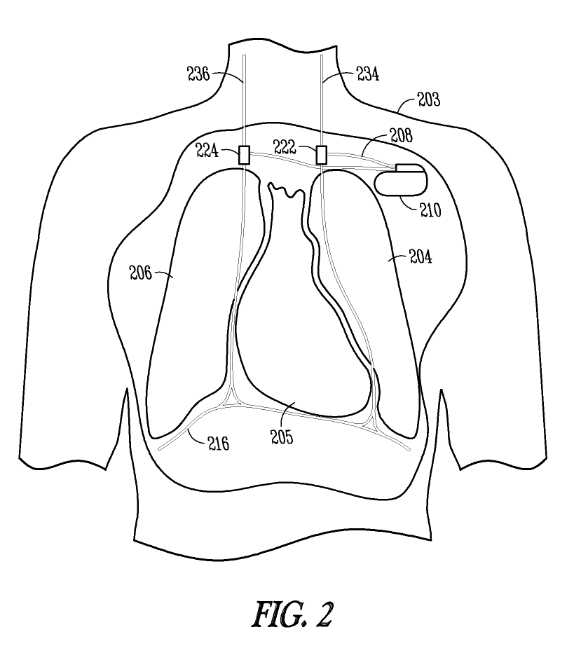

FIG. 2 is an illustration of an example of an IMD 210 implanted in a thorax region of a patient 203. The illustration shows the heart 205 of the subject as well as the left lung 204 and right lung 206. Also shown are representations of the left vagus nerve 234 and right vagus nerve 236. The IMD 210 is shown implanted in the pectoral region of the patient 203. In the example, the IMD 210 is coupled to one or more subcutaneous leads 208. In certain examples, the lead 208 includes one or more over-the-nerve collars 222 and 224 containing electrodes for contacting a vagus nerve. In certain examples, the lead 208 includes one or more patch electrodes for contacting a vagus nerve. In certain examples, the lead 208 is a transvenous lead. The transvenous lead is placed in a vein in proximity to the vagus nerve to stimulate the vagus nerve. The IMD 210 provides electrical stimulation to either the right or left vagus nerve or both the right and left vagus nerve to artificially induce at least one of deep ventilation or rapid ventilation. In some examples, the IMD 210 includes such leads and electrodes dedicated to stimulating the phrenic nerves instead of, or in addition to, the vagus nerves.

As stated previously, an IMD can be used to directly stimulate the subject's diaphragm. In FIG. 2, note that the apex of the right ventricle of the heart 205 is located close to the diaphragm 216 of the patient 203. Returning to FIG. 1, the RV tip electrode 120A that is positioned in the apex of the RV can be used to provide the direct stimulation to the diaphragm. The apical placement is proximate the diaphragm and electrical stimulation can stimulate the diaphragm when electrical energy is provided to the electrode. In some examples, an implantable lead can be fed through a thoracic duct of the lymphatic system to make direct contact with the diaphragm. Descriptions of using such an implantable lead to contact the diaphragm can be found in Brooke et al, “Method and Apparatus for Sensing Respiratory Activities Using Sensor in Lymphatic System,” U.S. Patent Pub. No. 20080234556, filed Sep. 25, 2008, which is incorporated by reference herein in its entirety.

The electrical stimulation energy used to artificially induce deep or rapid ventilation by stimulating one or more of a phrenic nerve, a vagus nerve, or the diaphragm is designed to avoid also causing a depolarization of cardiac tissue. The stimulation energy is provided in such a way to induce the ventilation but not induce cardiac depolarization. This stimulation energy is sometimes called sub-threshold energy.

In some examples, the frequency of the stimulation energy is high enough to avoid causing cardiac depolarization. In certain examples, the frequency of the stimulation energy is about fifty hertz (50 Hz). In some examples, the pulse width of the stimulation energy is made small enough to avoid causing depolarization. In certain examples, the pulse width is about 0.3 milliseconds (msec) and the amplitude of the stimulation energy is in the range of about five to six volts.

In some examples, the stimulation energy is provided during a myocardial refractory period. The myocardial refractory period closely follows a cardiac depolarization and is a time period when it takes a large amount of energy to cause a subsequent depolarization. Providing the stimulus during the refractory period allows energy of amplitudes and pulse widths to be used which would normally stimulate the heart outside the refractory period. In certain examples, the stimulation energy includes a pulse width of thirty milliseconds (30 ms). In certain examples, the stimulation energy includes amplitudes in the range of about eight to twelve volts.

As stated previously, an IMD can be used to selectively stimulate the subject's thoracic or chest muscles to enhance inhalation after an artificially induced gasp or cough. In some examples, a stimulation vector that includes a shock electrode (e.g., an SVC coil electrode or a RV coil electrode) can be used to deliver a low amplitude high frequency pulse train to selectively stimulate thoracic muscles to enhance inhalation. In some examples, a combination of stimulation electrodes can be used to stimulate a combination of phrenic nerves, vagus nerves, the diaphragm, and the thoracic muscles. For instance, a combination of electrodes can be used to simultaneously stimulate the phrenic nerves, the diaphragm, and the thoracic muscles. Such a combination is useful to push blood flows out of the thorax region and into the brain region of the patient. Again, in some examples, an IMD is programmed to scan through different vectors to find the vector that induces the strongest desired effect.

FIG. 3 shows a flow diagram of an example of a method 300 of artificially inducing ventilation in a subject using an IMD. At block 305, an episode of reduced hemodynamic capacity is detected in the subject using an IMD. The episode may include one or more of a breathing disorder, an episode of hemodynamic compromise, or a cardiac arrhythmia such as tachyarrhythmia.

At block 310, at least one of rapid or deep ventilation is artificially induced in the subject using electrical stimulation energy provided by the IMD in response to detecting the episode of reduced hemodynamic capacity. An example of such ventilation includes at least one of a cough or gasp. At block 315, a measure of hemodynamic performance is obtained after the electrical stimulation is provided. In some examples, the measure of hemodynamic performance is obtained after the IMD determines that the stimulation energy did artificially induce a cough or gasp. For instance, the IMD may include an activity sensor such as an accelerometer to detect action of thoracic muscles or to determine the strength of the cough or gasp.

The measure of hemodynamic performance may include, among other things, a measure of blood pressure, a measure of blood oxygenation, a measured heart sound parameter, a measured depolarization parameter, a measure of blood flow, provided by one or more sensors. The measure of hemodynamic performance is useful in providing feedback in determining the efficacy of the artificially induced ventilation. In response to the measure, the IMD may adjust the resuscitation therapy, such as by changing one or more of an amplitude of the stimulation energy, the pulse width of the stimulation energy, the frequency of the stimulation energy, or a combination of electrodes used to provide the stimulation.

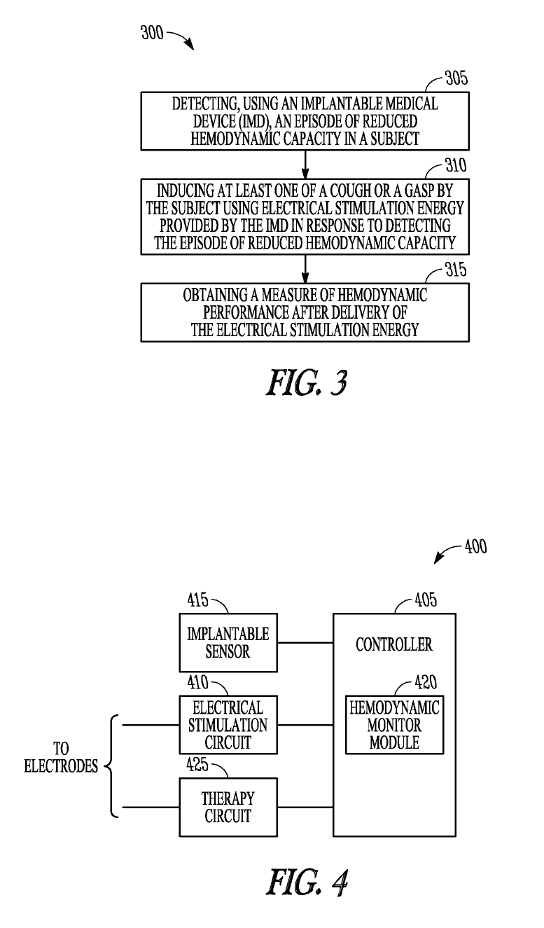

FIG. 4 shows a block diagram of portions of an example of a device 400 that artificially induces ventilation in a subject. The device 400 includes a controller 405, an electrical stimulation circuit 410, and at least one implantable sensor 415. The implantable sensor 415 provides a sensor signal representative of hemodynamic function of the subject. As stated previously, such a sensor includes, among other things, a cardiac signal sensing circuit, an intracardiac impedance sensing circuit, a transthoracic impedance sensing circuit, a blood pressure sensor, a blood flow sensor, a blood gas sensor, a chemical sensor, a heart sound sensor, a posture sensor, and an activity sensor.

The electrical stimulation circuit 410 provides electrical stimulation energy to an implantable electrode. The stimulation energy is deigned to artificially induce deep or rapid ventilation, such as a cough or gasp, in the subject. In certain examples, the controller 405 includes a processor, such as microprocessor, a digital signal processor, or other kind of processor. In certain examples, the controller 405 is an application specific integrated circuit (ASIC). In certain examples, the controller 405 performs instructions embodied in software, firmware, or hardware. In certain examples, the controller includes a sequencer that proceeds through logic functions implemented by hardware. To perform the functions described herein, the controller 405 may include modules. Modules can be hardware, firmware, or software, or any combination of hardware, firmware, and software. One or more functions may be performed by one module.

The controller 405 is communicatively coupled to the implantable sensor 415 and the stimulation circuit 410. The communicative coupling allows the controller 405 to transmit and/or receive signals to or from the stimulation circuit 410 and the implantable sensor 415 even though there may be intervening circuitry. The controller 405 includes a hemodynamic monitor module 420 that detects an episode of reduced hemodynamic capacity in a subject using the sensor signal provided by the implantable sensor 415.

In some examples, the implantable sensor 415 includes a cardiac signal sensing circuit configured to provide a sensed cardiac signal representative of cardiac depolarization events of the subject, and the hemodynamic monitor module 420 detects an episode of reduced hemodynamic capacity that includes cardiac arrhythmia from the sensed depolarization events. In some examples, the hemodynamic monitor module 420 detects arrhythmia using an assessment of heart rhythm stability when a subject experiences a sudden increase in depolarization rate. Examples of methods and systems to detect abnormal heart rhythms and assess the stability of the rhythms are found in Gilkerson et al., U.S. Pat. No. 6,493,579, entitled “System and Method for Detection Enhancement Programming,” filed Aug. 20, 1999, which is incorporated herein by reference.

In some examples, the implantable sensor 415 includes an implantable respiration sensor that provides a sensor signal representative of respiration. The hemodynamic monitor module 420 detects an episode of reduced hemodynamic capacity that may include a decrease in respiration tidal volume from the sensor signal. An example of an implantable respiration sensor is a transthoracic impedance sensor to measure minute respiration volume. An approach to measuring transthoracic impedance is described in Hartley et al., U.S. Pat. No. 6,076,015 “Rate Adaptive Cardiac Rhythm Management Device Using Transthoracic Impedance,” filed Feb. 27, 1998, which is incorporated herein by reference in its entirety.

In some examples, the second implantable sensor includes a blood flow sensor that provides a sensor signal representative of patient's blood flow. The hemodynamic monitor module 420 detects, using the sensor signal, an episode of reduced hemodynamic capacity that includes an onset of hemodynamic compromise. Examples of a blood flow sensor include a cardiac output sensor circuit or a stroke volume sensor circuit. Examples of stroke volume sensing are discussed in Salo et al., U.S. Pat. No. 4,686,987, “Biomedical Method And Apparatus For Controlling The Administration Of Therapy To A Patient In Response To Changes In Physiologic Demand,” filed Mar. 29, 1982, and in Hauck et al., U.S. Pat. No. 5,284,136, “Dual Indifferent Electrode Pacemaker,” filed May 13, 1991, which are incorporated herein by reference in their entirety.

In response to the detected episode of reduced hemodynamic capacity, the controller 405 initiates delivery of the electrical stimulation energy to artificially induce at least one of deep ventilation or rapid ventilation in the subject. According to some examples, the device 400 includes an implantable electrode communicatively coupled to the stimulation circuit 410. The implantable electrode is configured by shape and size for placement on or near a phrenic nerve or a vagus nerve. For instance, the implantable electrode may be included in an implantable lead. The controller 405 initiates delivery of the electrical stimulation energy to the phrenic nerve or the vagus nerve to artificially induce the ventilation. In certain examples, the device includes multiple electrodes for placement on both phrenic nerves, or on both vagus nerves, or on or near at least phrenic nerve and at least one vagus nerve. The controller 405 initiates delivery of the stimulation energy to the multiple electrodes.

According to some examples, the implantable electrode is configured for placement on or near a diaphragm of the subject. The controller 405 initiates electrical stimulation of the diaphragm to artificially induce the ventilation. In some examples, the implantable electrodes include a shock electrode (e.g., an SVC coil electrode or a RV coil electrode). The controller 405 initiates delivery of stimulation energy to the electrodes to selectively stimulate thoracic muscles to enhance inhalation.

After delivery of the electrical stimulation energy, the hemodynamic monitor module 420 obtains a measure of hemodynamic performance after delivery of the electrical stimulation to induce the artificial hyperventilation. Examples include a measure of arterial pressure, a measure of cardiac stroke volume, a measure of coronary perfusion, and a measure of cerebral perfusion. The measurement provides feedback to the controller 405 as to the effect of the deep ventilation or rapid ventilation on the hemodynamic system. The depth of a cough or gasp may be correlated to cerebral perfusion pressure. Thus, a deeper cough or gasp betters the improvement in the hemodynamic system.

If the feedback indicates that the artificially induced ventilation was inadequate to improve hemodynamic capacity, the controller 405 may induce additional ventilation, such as another cough or gasp for example. In certain examples, if the controller 405 deems that the effect of the artificially induced ventilation was inadequate, the controller 405, in response to the measure of hemodynamic performance, may adjust the electrical stimulation energy to change an intensity of the ventilation. The controller 405 may change the intensity by adjusting one or more of the magnitude of the stimulation energy, the frequency of the stimulation energy, and pulse width of the stimulation energy. In certain examples, the controller 405 selects an alternate implantable electrode or electrodes to deliver the electrical stimulation energy, or both adjusts the electrical stimulation energy and selects alternate implantable electrodes.

In some examples, the sensor signal provided by the implantable sensor 415 is used to detect the episode of reduced hemodynamic capacity and is used to determine the measure of hemodynamic performance after delivery of the electrical stimulation energy. For instance, after detecting an episode of reduced hemodynamic capacity and after the stimulation is delivered to artificially induce a cough or gasp, the hemodynamic monitor module 420 may determine, using a sensor signal provided by a blood flow sensor, a measure of coronary or cerebral perfusion.

In another example, after detecting an arrhythmia and after the stimulation is delivered to artificially induce the ventilation, the hemodynamic monitor module 420 may determine from the sensed cardiac signal that the arrhythmia self-terminated, or may determine that the arrhythmia persists after the deep or rapid ventilation.

In some examples, different sensor signals are used by the hemodynamic monitor module 420 to detect the episode of reduced hemodynamic capacity and to obtain the measure of hemodynamic performance. Different sensors provide the sensor signals. For instance, the device 400 may include a second implantable sensor such as an arterial pressure sensor. The measure of hemodynamic performance may include a measure of arterial pressure obtained using a sensor signal provided by the arterial pressure sensor.

In certain examples, the arterial pressure sensor is an implantable pulmonary arterial (PA) pressure sensor. Such a sensor is useful to detect a reduction in blood supply to a portion of the heart. To detect the reduction in blood supply, at least one feature of the PAP signal is identified. Examples of the identifiable feature include, among other things, at least one detected amplitude, at least one detected magnitude, at least one detected peak, at least one detected valley, at least one detected value, at least one detected change, at least one detected increase, at least one detected decrease, and at least one detected rate of change in the at least one PA pressure characteristic. The time interval between two occurrence of the identifiable feature is then determined. The feature and the time interval between two occurrences of the feature can be identified by using a signal processor.

One or more time intervals may be used to compute an indication of a reduction of blood supply to at least a portion of a heart. As an example, if the identifiable feature is a magnitude of PA end-diastolic pressure (“PAEDP”), a 25% reduction of blood supply to at least a portion of the heart can be computed if the interval between a detected PAEDP magnitude having a first level and a detected PAEDP magnitude having a second level that exceeds the first level by a certain amount (e.g., 50 mmHg) occurs within a certain amount of time (e.g., 45 seconds). An approach for detecting a reduction in blood supply to a portion of the heart using PA pressure is described in Zhang et al., commonly assigned, co-pending, U.S. patent application Ser. No. 11/624,974, entitled “Ischemia Detection Using Pressure Sensor,” filed Jan. 19, 2007, which is incorporated herein by reference.

In another example, the device 400 may include a second implantable sensor such as an intracardiac impedance sensor. Electrodes placed within a chamber of the heart provide a signal of intracardiac impedance versus time. The electrodes may be placed in a right ventricle of the heart and the measured intracardiac impedance waveform can be signal processed to obtain a measure of the time interval beginning with a paced or spontaneous QRS complex (systole marker) and ending with a point where the impedance signal crosses the zero axis in the positive direction following the QRS complex. The resulting time interval is inversely proportional to the contractility of the heart. Systems and methods to measure intracardiac impedance are described in Citak et al., U.S. Pat. No. 4,773,401, entitled “Physiologic Control of Pacemaker Rate Using Pre-Ejection Interval as the Controlling Parameter,” filed Aug. 21, 1987, which is incorporated herein by reference in its entirety. The hemodynamic monitor module 420 is configured to obtain a measure of cardiac stroke volume using a sensor signal provided by the intracardiac impedance sensor.

In still another example, the device 400 may include at least one of an activity sensor (e.g., an accelerometer) that provides a sensor signal representative of chest muscle activity, and a transthoracic impedance sensor to provide a sensor signal representative of transthoracic impedance. The controller 405 determines the intensity or strength of the artificially induced ventilation using the provided sensor signal. Based on the determined intensity, the controller 405 may adjust the electrical stimulation energy to induce the rapid ventilation or deep ventilation, select alternate implantable electrodes to deliver the stimulation energy, or may both adjust the electrical stimulation energy and select alternate implantable electrodes.

According to some examples, the device 400 is a CFM device. The implantable sensor 415 includes a cardiac signal sensing circuit configured to provide a sensed cardiac signal representative of cardiac depolarization events of the subject. The CFM device may include a therapy circuit 425. The therapy circuit 425 is communicatively coupled to implantable electrodes located in the heart or in proximity of the heart to provide electrical cardiac therapy. In some examples, the therapy circuit 425 provides anti-tachyarrhythmia therapy. In certain examples, the therapy circuit 425 provides at least one of anti-tachycardia pacing (ATP), cardioversion shock therapy, or defibrillation shock therapy. In some examples, the therapy circuit 425 includes different charging capacitors than the electrical stimulation circuit 410.

In some examples, the stimulation circuit 410 is communicatively coupled to the same implantable electrodes as the therapy circuit 425 (e.g., to RV lead electrodes, LV lead electrodes in FIG. 1). In some examples, the stimulation circuit 410 is communicatively coupled to different implantable electrodes from the therapy circuit 425. For instance, the therapy circuit 425 may be coupled to one or more of the lead electrodes or electrodes formed on the can in FIG. 1, and the stimulation circuit may be coupled to electrodes dedicated for nerve stimulation such as one or more of the electrodes in FIG. 2. In another example, the therapy circuit 425 may be coupled to one or more of the lead electrodes or electrodes formed on the can in FIG. 1 and the stimulation circuit may be coupled to electrodes dedicated for direct stimulation of the diaphragm.

The hemodynamic monitor module 420 is configured to detect an episode of tachyarrhythmia using the sensed cardiac signal. In some examples, the hemodynamic monitor module 420 is configured to detect an episode of tachyarrhythmia using the sensed cardiac signal. In certain examples, the hemodynamic monitor module 420 detects an episode of tachyarrhythmia by detecting a sudden increase in a heart depolarization rate that exceeds a specified heart rate detection threshold. In certain examples, once the heart rate detection threshold is exceeded, other detection methods may be used to confirm that a patient is indeed experiencing tachyarrhythmia. For instance, the hemodynamic monitor module 420 may detect tachyarrhythmia using the previously mentioned assessment of heart rhythm stability when a subject experiences a sudden increase in depolarization rate.

When the episode of tachyarrhythmia is detected, the controller 405 initiates delivery of electrical stimulation energy to artificially induce the deep and/or rapid ventilation prior to initiating anti-tachyarrhythmia therapy. If the anti-tachyarrhythmia therapy includes shock therapy, this artificial hyperventilation may be useful to improve efficacy of the shock therapy.

Deep gasps from artificially induced ventilation provide a chance for more oxygenation and catecholamine circulation in the blood just prior to the shock. This additional oxygenation and catecholamine circulation may serve to prime the myocardium to improve the effectiveness of the shock and aid in post-shock recovery. During tachyarrhythmia, although there is a net increase in sympathetic nerve tone, anatomical innervations of the heart are highly heterogeneous; some areas of the heart are highly innervated while others have little to no sympathetic nerve innervation. Additional catecholamine circulation in the blood may provide a more global effect on the heart to likely reduce subsequent reentrant arrhythmias resulting from heterogeneous stimulation of adregenic receptors.

Additionally, the deep gasps from artificially induced ventilation may provide some stabilization of the hemodynamic system. This stabilization gives more time for ATP therapy to convert a rhythm before resorting to shock therapy, if necessary, without compromising the efficacy of the shock therapy. Further, this stabilization provides more time for the tachyarrhythmia to self-terminate. This may result in less delivery of anti-arrhythmia therapy, thereby preserving battery life of the implantable device and improving patient comfort. Further still, this hemodynamic stabilization provides more time for the patient to take any actions before the shock is delivered, such as lying down or pulling her car over to the side of the road in anticipation of the shock.

In some examples, the controller 405 directly initiates delivery of the anti-tachyarrhythmia therapy after delivery of the electrical stimulation energy. In some examples, the hemodynamic monitor module 420 first reconfirms the tachyarrhythmia after the electrical stimulation energy is delivered prior to the initiation of the tachyarrhythmia therapy. This may also result in less delivery of anti-tachyarrhythmia therapy. In certain examples, tachyarrhythmia is reconfirmed using methods similar to the original detection and confirmation of the tachyarrhythmia.

According to some examples, the controller 405 initiates the delivery of electrical stimulation energy to artificially induce the deep and/or rapid ventilation after anti-tachyarrhythmia therapy is delivered. In some examples, the anti-tachyarrhythmia therapy includes shock therapy. Artificially inducing a cough or gasp after shock therapy is delivered may help in converting the arrhythmia to normal sinus rhythm (NSR). In certain examples, the controller 405 initiates the delivery of electrical stimulation energy to artificially induce a cough or gasp when the tachyarrhythmia is sustained after delivery of a number of shocks that exceeds a threshold number of shock therapy deliveries. The threshold number may be a total count of the number of shocks or a number of shocks given within a specified duration of time. In certain examples, the controller 405 initiates the delivery of electrical stimulation energy to induce a cough or gasp when the tachyarrhythmia (e.g., VT or VF) is sustained for a specified duration of time.

After delivery of anti-tachyarrhythmia shock therapy, a patient may experience post-shock hypotension or malignant vasovagal syncope. Artificially inducing one or both of deep ventilation and rapid ventilation may improve oxygenation to relieve the hypotension or syncope. In some examples, the device 400 includes one or more of a blood pressure sensor and a respiration sensor. The hemodynamic monitor module 420 detects hypotension or syncope by detecting one or more of low blood pressure or shortness of breath. In response, the controller 405 initiates delivery of electrical stimulation energy to induce artificial hyperventilation.

Further, after delivery of anti-tachyarrhythmia shock therapy a patient's heart may exhibit pulse-less electrical activity (PEA). This is when electrical impulses are present in the heart, but they do not produce depolarization. Artificial inducement of a cough or gasp may relieve PEA. In some examples, the device 400 includes a cardiac signal sensing circuit and the hemodynamic monitor module 420 detects after-shock PEA using a cardiac signal that is sensed post-shock. The controller 405 initiates delivery of electrical stimulation energy to induce a cough or gasp in response to the detected PEA.

Providing resuscitation therapy by an implantable medical device is helpful to the patient at times of reduced hemodynamic capacity, especially to minimize risk of hypotension and syncope. Tuning a cough or gasp of the resuscitation therapy to optimize the desired depth of the gasp or cough maximizes the improvement to the patient's hemodynamic function.

### Additional Notes

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
an implantable sensor configured to provide a sensor signal representative of hemodynamic function of a subject;
a stimulation circuit configured to provide electrical simulation energy to an implantable electrode; and
a controller communicatively coupled to the stimulation circuit and the implantable sensor and including a hemodynamic monitor module configured to detect an episode of reduced hemodynamic capacity in a subject using the sensor signal,
wherein the controller is configured to initiate delivery of the electrical stimulation energy to artificially induce at least one of a deep ventilation or a rapid ventilation by the subject in response to the detected episode, and
wherein the hemodynamic monitor module is configured to obtain a measure of hemodynamic performance after delivery of the electrical stimulation energy.

2. The apparatus of claim 1, including an implantable electrode communicatively coupled to the electrical stimulation circuit, wherein the implantable electrode is configured for placement on or near at least one of a phrenic nerve or a vagus nerve, and wherein the controller is configured to initiate electrical stimulation of the phrenic nerve or the vagus nerve to artificially induce one or both of the deep ventilation and rapid ventilation.

3. The apparatus of claim 1, including an implantable electrode communicatively coupled to the electrical stimulation circuit, wherein the implantable electrode is configured for placement on or near a diaphragm of the subject, and wherein the controller is configured to initiate delivery of electrical stimulation energy to the diaphragm to artificially induce one or both of the deep ventilation and rapid ventilation.

4. The apparatus of claim 1, wherein the controller is configured to initiate delivery of the electrical stimulation energy to artificially induce at least one of a cough or gasp by the subject in response to the detected episode.

5. The apparatus of claim 1, including a therapy circuit communicatively coupled to the controller and configured to provide anti-tachyarrhythmia therapy that includes at least one of:
anti-tachycardia pacing (ATP);
cardioversion shock therapy; or
defibrillation shock therapy; and
wherein the implantable sensor includes a cardiac signal sensing circuit configured to provide a sensed cardiac signal representative of cardiac depolarization events of the subject,
wherein the hemodynamic monitor module is configured to detect an episode of tachyarrhythmia using the sensed cardiac signal, and
wherein the controller is configured to initiate electrical stimulation to artificially induce one or both of the deep ventilation and the rapid ventilation prior to initiating anti-tachyarrhythmia therapy.

6. The apparatus of claim 5, wherein the hemodynamic monitor module is configured to reconfirm the tachyarrhythmia after the electrical stimulation to artificially induce the deep ventilation or rapid ventilation is delivered and prior to the initiation of the tachyarrhythmia therapy.

7. The apparatus of claim 1, including a therapy circuit communicatively coupled to the controller and configured to provide anti-tachyarrhythmia therapy that includes at least one of:
anti-tachycardia pacing (ATP);
cardioversion shock therapy; or
defibrillation shock therapy; and
wherein the implantable sensor includes a cardiac signal sensing circuit configured to provide a sensed cardiac signal representative of cardiac depolarization events of the subject,
wherein the hemodynamic monitor module is configured to detect an episode of tachyarrhythmia using the sensed cardiac signal, and
wherein the controller is configured to initiate the electrical stimulation to artificially induce the deep ventilation or rapid ventilation after anti-tachyarrhythmia therapy is delivered.

8. The apparatus of claim 7, wherein the controller is configured to initiate the electrical stimulation to artificially induce one or both of the deep ventilation and rapid ventilation when at least one of:
the episode of tachyarrhythmia is sustained for a time duration that exceeds a specified time duration threshold; or
the anti-tachyarrhythmia therapy includes shock therapy, and the tachyarrhythmia is sustained after delivery of a number of shocks that exceeds a threshold number of shock therapy deliveries.

9. The apparatus of claim 1, including at least one of:
an activity sensor configured to provide a sensor signal representative of chest muscle activity; and
a transthoracic impedance sensor to provide a sensor signal representative of transthoracic impedance, and
wherein the controller is configured to determine an intensity of the induced ventilation using the provided sensor signal and to adjust the electrical stimulation energy to artificially induce one or both of the deep ventilation and rapid ventilation according to the determined intensity.

10. The apparatus of claim 1, wherein the controller is configured to, in response to the measure of hemodynamic performance,
adjust the electrical stimulation to change an intensity of one or both of the deep ventilation and the rapid ventilation; or
select an alternate implantable electrode to deliver the electrical stimulation.

11. The apparatus of claim 1, wherein the hemodynamic monitor module is configured to detect, using the sensor signal provided by the implantable sensor, at least one of:
a decrease in respiration tidal volume; or
an onset of hemodynamic compromise.

12. A method comprising:
detecting, using an implantable medical device (IMD), an episode of reduced hemodynamic capacity in a subject;
artificially inducing at least one of a deep ventilation or a rapid ventilation in the subject using electrical stimulation energy provided by the IMD in response to detecting the episode of reduced hemodynamic capacity; and
obtaining a measure of hemodynamic performance after delivery of the electrical stimulation energy.

13. The method of claim 12, including, according to the measure of hemodynamic performance, at least one of:
adjusting the electrical stimulation provided by the IMD to change the strength of one or both of the deep ventilation and the rapid ventilation; or
changing at least one electrode used in providing the electrical stimulation.

14. The method of claim 12,
wherein detecting an episode of reduced hemodynamic capacity includes detecting an episode of tachyarrhythmia,
wherein artificially inducing the deep ventilation or rapid ventilation includes inducing one or both of the deep ventilation and rapid ventilation prior to providing anti-tachyarrhythmia therapy with the IMD, and
wherein the anti-tachyarrhythmia therapy includes at least one of:
anti-tachycardia pacing (ATP);
cardioversion shock therapy; or
defibrillation shock therapy.

15. The method of claim 14, including reconfirming the tachyarrhythmia after artificially inducing one or both of the deep ventilation and rapid ventilation and prior to providing the anti-tachyarrhythmia therapy.

16. The method of claim 12,
wherein detecting an episode of reduced hemodynamic capacity includes detecting an episode of tachyarrhythmia, and
wherein artificially inducing the deep ventilation or rapid ventilation includes artificially inducing one or both of the deep ventilation and rapid ventilation after providing anti-tachyarrhythmia therapy with the IMD.

17. The method of claim 16, wherein artificially inducing deep or rapid ventilation after providing anti-tachyarrhythmia therapy includes artificially inducing one or both of the deep or rapid ventilation when at least one of:
the episode of tachyarrhythmia is sustained for a time duration that exceeds a specified time duration threshold, or
the anti-tachyarrhythmia therapy includes shock therapy, and the tachyarrhythmia is sustained after delivery of a number of shocks that exceeds a threshold number of shock therapy deliveries.

18. The method of claim 12, wherein artificially inducing deep ventilation or rapid ventilation includes artificially inducing at least one of a cough or gasp by the subject.

19. The method of claim 12, including:
determining an intensity of the artificially induced deep ventilation or rapid ventilation; and
adjusting the electrical stimulation provided by the IMD to change the intensity of one or both of the deep ventilation and the rapid ventilation.

20. The method of claim 12, wherein obtaining a measure of hemodynamic performance includes obtaining a measure of at least one of:
arterial pressure;
cardiac stroke volume;
coronary perfusion; or
cerebral perfusion.
